(19) United States
(12) Patent Application Publication
(10) Pub. No.: US 2020/0306470 A1
(43) Pub. Date: Oct. 1, 2020

(54) BREATHING SYSTEM COMPONENT AND A PROCESS FOR THE MANUFACTURE OF THE BREATHING SYSTEM COMPONENT

(71) Applicant: MAQUET CRITICAL CARE AB, Solna (SE)

(72) Inventor: Johan REUTERHOLT, Bålsta (SE)

(21) Appl. No.: 16/900,817

(22) Filed: Jun. 12, 2020

Related U.S. Application Data
(63) Continuation-in-part of application No. PCT/SE2018/051300, filed on Dec. 12, 2018.

(30) Foreign Application Priority Data
Dec. 15, 2017 (WO) PCTSE2017051284

(51) Int. Cl. A61M 16/00; A61L 31/04; A61L 31/14

(52) U.S. Cl. CPC A61M 16/0057; A61L 31/048; A61M 16/0833; A61M 2207/00; A61L 31/141

(57) ABSTRACT

The present disclosure relates to a breathing system component (25) comprising a hollow gas duct (50) for conveying breathing gas in the breathing system (1) and to a process for the manufacture of the breathing system component. The component has an integrated one-piece molded structure and is made of a non-toxic plastic material having a melt flow rate MFR of 2.5 g/10 min or less, measured according to ISO 1133 with a load of 2.16 kg at a temperature of 230° C.

This application is a bypass application under 35 U.S.C. § 111(a) and claims benefit of, and priority to, International Application No. PCT/SE2018/051300 filed on Dec. 12, 2018, which claims priority to International Application No. PCT/SE2017/051284 filed on Dec. 15, 2017. These above-mentioned International Applications are incorporated herein by reference in theft entirety for all they disclose.

### TECHNICAL FIELD

The present disclosure relates to a breathing system component and to a process for the manufacture of the breathing system component. The disclosure further relates to a reflector body, a reflector unit and a breathing system comprising the reflector body and/or the reflector unit.

### BACKGROUND ART

In the field of mechanical ventilation and breathing aid, there are different types of patient breathing systems used to deliver a desired gas mixture to a patient. The breathing system may be of a non-re-breathing or re-breathing type. Re-breathing systems are often used when expensive additive gases are administered to the patient besides the necessary life sustaining gas mixture. In re-breathing systems exhaled gas is re-supplied to the patient, wherein the additive gas not assimilated by the patient in previous inhalations may be absorbed during the following inhalation. Examples of additive gases are helium which may be used, e.g., in severe cases of asthma, xenon which may be used as contrast medium in diagnostic computer tomography (CT) treatment, and different anesthetic gases which may be used as additive in inhalation anesthesia. Breathing systems are generally discussed, for example, in WO2014041104, U.S. Pat. No. 8,186,347 and EP2168623.

Breathing systems generally comprise a ventilator, which is connected to a driving gas supply. The driving gas flow is used to generate patient inhalation and the driving gas may be, for example, oxygen or an air/oxygen mixture. The breathing system further comprises an inspiratory line and an expiratory line connected to the patient. The inspiratory and expiratory lines can be arranged in a closed breathing circuit or circle, also referred to as a patient circuit. A fresh gas supply arrangement is usually connected to the inspiratory line. Furthermore, the breathing circuit may comprise a carbon dioxide absorber. The breathing circuit may comprise check valves, compressors or ejectors to regulate the gas flow to a desired direction. Further, the breathing system suitably comprises an arrangement which separates the driving gas of the ventilator from the patient breathing gases in the patient circuit. The arrangement may be formed, for example, of a bag in bottle arrangement or an extended pathway extending between the ventilator and the patient circuit.

Components conveying patient gas or driving gas in the breathing systems have high demands with regard to patient safety. Thus, the components should have robust structure, which is easy to handle while the materials used should be non-toxic, sterilizable and tolerate mechanical impacts. Due to these high demands, the components are often manufactured of several sub-components, which are assembled together and the manufacturing processes are often complicated. Furthermore, expensive raw materials are included in the components. Even though the known components fulfil the requirements regarding the patient safety, there is a desire to simplify the structure of the components while still ensuring the components fulfil the requirements in respect of patient safety. Furthermore, there is a desire to simplify the manufacturing processes of the components.

### SUMMARY OF THE DISCLOSURE

A breathing system component adapted for conveying a breathing gas in the breathing system, especially a component adapted to be in fluid contact with a patient via a gas that may have a high humidity, such as a component having a gas duct for conveying a breathing gas to a patient, needs to comply with high patient safety demands. As mentioned above, such components have been manufactured to be robust and bio-compatible with respect to its intended use. Also, for use of the components in hospitals they have been made to tolerate cleaning and autoclave treatment. Thus, e.g., larger component structures may have been assembled of several separately sterilisable sub-components. The complicated structures have involved complicated manufacturing processes, which has led to both high costs and tedious manufacturing processes. For example, in a situation where a component needs to be replaced in an existing system, high costs and long delivery times may have been troublesome.

Therefore, there is a need to simplify the structure of the components and simplify the manufacturing process while the components still comply with the operational robustness requirements and patient safety.

In view of the problems above, it is an objective of the present disclosure to simplify the structure of breathing system components. It also an objective of the present disclosure to simplify manufacturing processes for breathing system components.

It also an objective of the present disclosure to provide a breathing system component which is robust and sterilisable.

Further, it is an objective of the present disclosure to provide a breathing system component that is easy to handle and that can be used in existing breathing systems.

The objectives above are attained by a breathing system component, breathing system, volume reflector unit and a process for the manufacture of a breathing system component, comprising a hollow gas duct for conveying an inspiratory gas to a patient as defined in the appended claims.

Accordingly, the present disclosure relates to a breathing system component comprising a hollow gas duct for conveying breathing gas in the breathing system. By breathing gas is meant in this application both inspiratory and expiratory gases as well as driving gas, which comprises oxygen. The component has an integrated one-piece molded structure and is made of a non-toxic plastic material having a melt flow rate (MFR) of 2.5 g/10 min or less, measured according to ISO 1133 with a load of 2.16 kg and at a temperature of 230° C., which may be abbreviated as 2.16 kg/230° C. It has been noted that breathing system components manufactured of the plastic material having the defined melt flow rate can be molded whereby a simplified integrated structure for the components can be obtained, while the components still comply with the operational robustness requirements and patient safety. Also, it is possible to simplify the manufacturing process.

Especially suitable plastic materials may have the melt flow rate MFR from 0.6 to 1.0 g/10 min, measured according to ISO 1133 with 2.16 kg/230° C.

The breathing system component may be molded by means of blow molding. In this way integrated one-piece molded structures having uniform material characteristics throughout the whole component can be obtained in a simple and effective way.

The plastic material may have a melting point adapted to be above a sterilization temperature of the breathing system component. The melting point may be at least 134° C., measured according to ASTM D3418-15. The melting point may be, for example, from 140 to 220° C., measured according to ASTM D3418-15. By having the melting point in these temperature intervals, it is possible to provide a re-usable breathing system component, which can be cleaned and sterilized several times.

The flexural modulus of the plastic material may be from 900 to 2000 MPa, preferably from 1000-1500 MPa, measured at 23° C. and according to ISO 178. In this way, the material has a sufficiently low tendency to bend, while it is still easy to handle.

The Izod impact of the plastic material may be at least 3, or from 3.5 to 10 kJ/m², measured according to ISO 180/1A at −20° C., according to a version valid at the priority date of the application, when the material is notched. In this way it can be assured that the breathing system component resists impacts during handling without breakage, whereby patient safety of the component may be further improved.

The density of the plastic material may be from 0.8 to 1.0 g/mL at 25° C. Thus, a light-weight product which is easy to handle may be provided.

The plastic material may be polyolefin-based. The polyolefin-based materials are non-toxic materials which are easy to use in e.g., blow molding processes. Suitably, the polyolefin-based material may be polyethylene, polypropylene or a copolymer thereof. These materials have good cold impact resistance and stiffness, are suitable for blow molding applications and can be steam steam-sterilized or autoclaved.

The hollow gas duct of the breathing system component may be a single continuous gas duct comprising straight and curved portions. Components of this type are suitable for conveying breathing gas in for example breathing gas separator units, e.g., in volume reflector bodies.

The hollow gas duct may be arranged such that at least two gas duct sections are located adjacent to each other and connected to each other by means of at least one solid portion. By placing gas duct portions adjacent to each other, the length of the gas duct may be increased. The gas duct may comprise free ends, wherein at one free end is formed a first port and at a second free end is formed a second port, and each of the first port and the second port comprises means to connect the breathing system component to the breathing system. Thus, the breathing system component may be connectable to various other components of the breathing system.

According to an embodiment, the hollow gas duct may resemble or have a shape of a folded tube such that the free ends of the tube are placed adjacent to each other and wherein the at least two adjacent gas duct portions run side-by-side, and wherein the tube is spirally wound inwards towards a centre point of the breathing system component. In this way, an increased gas duct length may be provided while the uptake area of the breathing system component will be minimized. The folded end of the tube may be arranged such that two central loops are formed in the centre portion of the breathing system component. In this way sharp folding edges can be prevented and the gas may be smoothly conveyed in the gas duct.

According to a variant, the component may have a substantially quadrilateral shape. By substantially quadrilateral shape is meant a principal shape of the component in an X-Y plane. Also quadrilateral shapes in which at least one, two, three or four edges have rounded shape are included in the definition. Also, the length of the sides in the quadrilateral shape may be the same or different. By providing the quadrilateral shape, the overall space required by the breathing system component in a breathing system may be minimized.

The breathing system component defined above may be a volume reflector body. Thus, sufficient length for the gas duct in the volume reflector body may be provided. The length of the gas duct in the volume reflector body may be from 0.5 to 4 m. The total gas duct volume may be from 0.1 to 2 litres. Thus, different sizes for the volume reflector body to fulfil different patient requirements may be provided in an easy and effective way.

The present disclosure also relates to a process for the manufacture of a breathing system component as described above. The method comprises the steps of:
a. providing a non-toxic plastic material having a melt flow rate MFR of 2.5 g/10 min or less, measured according to ISO 1133 with a load of 2.16 kg at a temperature of 230° C.;
b. providing a mold comprising a cavity having a shape corresponding to the shape of the outer contour of the breathing system component;
c. melting the non-toxic plastic material and providing the melted plastic material to the mold;
d. blow molding the breathing system component by means of inflating the melted plastic material with pressurized gas so that the melted plastic material is pressed towards the walls of the cavity in the mold; and
e. opening the mold and removing the blow-molded breathing system component.

Step c) may include a step of forming a parison of the molten plastic material. In that case, step d) may include a step of inflating the parison with pressurized gas. The parison may be formed by means of extrusion or it may be pre-formed by injection molding.

The process may further comprise cutting the blow molded breathing system component to remove excess material surrounding the edges of the component. Also, the breathing system component may comprise two free gas duct ends, and the process may further comprise machining the two free gas duct ends to provide sealing surfaces for additional components. In this way, the breathing system component will be easily connectable to the breathing system.

The present disclosure also relates to a breathing system component produced by the process as described above. By the process an integrated one-piece structure may be provided in an easy and effective way, while the breathing system component is robust and complies with patient safety requirements.

The present disclosure also relates to a volume reflector unit comprising the breathing system component that is the volume reflector body as described above and a carrier having a shape adapted to the shape of the volume reflector body. In this way the unit may be easily connected to an interface in a breathing system. The carrier, also referred to as a carrier means, may have edges adapted to at least partially surround the volume reflector body. In this way, the volume reflector body can be protected against outside forces.

The present disclosure further relates to a breathing system comprising a ventilator providing a driving gas flow, a patient circuit comprising inhalation and exhalation lines connectable to a patient, a fresh gas supply inlet connectable to the inhalation line, and an arrangement which separates the driving gas of the ventilator from the patient breathing gases in the patient circuit, wherein the system comprises the breathing system component as defined above.

Further features and advantages of embodiments according to the present disclosure will be defined more in detail in the detailed description below with reference to the appended drawings.

### BRIEF DESCRIPTION OF THE DRAWINGS

FIG. 1 schematically illustrates a breathing system comprising a breathing system component of the present disclosure;

FIG. 2 schematically illustrates an example embodiment of a breathing system component of the present disclosure in a perspective view from above;

FIG. 3 schematically illustrates an example embodiment of a breathing system component of the present disclosure from below;

FIG. 4 schematically illustrates an example embodiment of a volume reflector unit in a perspective view;

FIG. 5 illustrates the steps of the manufacturing process according to the present disclosure.

### DETAILED DESCRIPTION

The breathing system component of the present disclosure is made of a non-toxic plastic material, since the component is to be used in fluid contact with a patient. By “made of” is in this application meant that the integrated one-piece molded structure mainly comprises or consists of the non-toxic plastic material, which is suitably bio-compatible. However, the molded structure may be provided with or connected to other components of the system made of other materials.

By non-toxic material is meant a material which is not toxic and can be used in direct or indirect contact with humans. Examples of non-toxic plastic materials suitable for the component are food-grade or medical-grade plastic materials.

By “integrated one-piece molded structure” is meant that the breathing system component is molded and has a unitary construction and is thus not assembled together of several sub-components which are fastened together by mechanical means. The structure may be formed by molding it in one piece, whereby a hollow gas duct for conveying a breathing gas which may comprise an inspiratory, expiratory and/or driving gas is formed. The molding process may be blow molding, also referred to as blow forming, whereby a one-piece structure is readily obtained. The formed one-piece component can be connected to or arranged in connection with other components in the breathing system.

Also the plastic material may be chosen so that patient gases comprising, e.g., anaesthetic agents, are not absorbed or adsorbed by the material. This is important in case the component is re-used in a breathing system.

Breathing system components have specific requirements for complying with the patient safety. Therefore, the structure of the previously known breathing system components, such as volume reflectors, has been robust and often complicated. The components have often been assembled together of several sub-components to obtain the final structure of the breathing system component. Also, it has been a requirement that the components can withstand autoclaving or sterilizing at high temperatures. Therefore, it has previously not been considered feasible to manufacture and use one-piece integrated structures produced by molding processes, and in particular blow-molding process, for the breathing system components. However, it has now been discovered that non-toxic plastic materials having a specific melt flow rate are suitable for molding breathing gas components that comply with the requirements for patient safety. The non-toxic plastic materials are preferably bio-compatible. The melt flow rate (MFR) is measured according to a standard ISO 1133. More specifically a version ISO 1133-1:2011, which was valid at the priority date of the application, can be used. The melt flow rate can be up to and including 2.5 g/10 min, measured according to ISO 1133-1:2011 with 2.16 kg/230° C., or up to and including 6 g/10 min, or 5.5 or 5 g/10 min, measured according to ISO 1133-1:2011. Alternative method for measuring could be used, e.g. ASTM D1238 with 5.00 kg/230° C. The ISO- and ASTM-methods are similar, and in the methods the melt flow rate is determined by extruding molten polymeric material and by measuring the mass of the polymer flowing in ten minutes through a barrel of a plastometer. The temperature and the load are pre-determined and in the measurements, in the method according to ISO 1133-1:2011 the used load is 2.16 kg and the temperature 230° C. In this way, the material has a property that makes it possible to, e.g., hold a parison for the required thickness and breathing system component geometry during a blow-molding process. Therefore, it is possible to provide an integrated one-piece structure for the breathing system component, while the patient safety requirements are fulfilled.

Alternatively, the MFR range may be from 0.5 to 2.0 g/10 min, or from 0.5 to 1.5 g/10 min, or from 0.6 to 1.0 g/10 min, measured according to ISO 1133-1:2011 with 2.16 kg/230° C., whereby also more complicated shapes of the component may be blow-molded. According to an aspect, the MFR may vary from 0.6 to 1.0 g/10 min, whereby manufacturing is further facilitated. According to a further aspect, the MFR varies from 0.65 to 0.95 g/10 min, measured according to ISO 1133-1:2011 with 2.16 kg/230° C., whereby complicated structures can be effectively manufactured by means of blow-molding.

Non-toxic plastic materials having the specific melt flow rate are commercially available on the market and may be provided, e.g., by a company Ineos® or Kolon Plastics Inc.®, but are not limited to these examples. The plastic material may be any plastic material that is non-toxic, moldable or blow-moldable and has the MFR within the specified range.

Also, due to different requirements in different hospitals and countries, the component may be adapted to withstand cleaning and/or sterilization before it is taken into use in breathing systems. A final step of cleaning often occurs in an autoclave at a maximum temperature of about 103-140° C., such as 134° C. Therefore, the material used in the breathing system component needs to tolerate short-term exposures to high temperatures. Thus, the plastic material may suitably have a melting point adapted to be above the sterilization temperature of the component and may be greater than 134° C., e.g., from 140 to 220° C., measured according to ASTM D3418-15. The melting point can be measured according to other suitable methods, such as ISO 11357, e.g. a version ISO 11357-3:2011, which was valid at the priority date of the application. The measurement is performed by means of a differential scanning calorimetry (DSC) and corresponds to peak DSC melting temperature. The heating rate which is normally used is 10° C./min.

The component may be single-use or re-usable. In case of a single use component it may be provided to the user clean and sterilized and ready to use. Alternatively, the component needs to be cleaned by the user before it is taken to use. However, after use the component it can be thrown away. If the component is re-usable it is desirable that it tolerates repeated autoclaving occasions, and at least 2 or more occasions, e.g., according to some embodiments it is possible that the material tolerates more than 5-10 cleaning cycles and thus autoclaving occasions. Upper limit of cleaning cycles is not critical, but may be set to, for example, 30 cleaning cycles. However, it is sufficient if the material withstands a short-time exposing to autoclaving temperatures.

Furthermore, according to the present disclosure it has been surprisingly noted that also complicated component shapes may be blow molded, if the melt flow rate of the plastic material is within a certain range. The plastic material usable according to the present disclosure is suitable to use for blow-molding both simple shapes and specific shapes of a breathing system, such as a reflector body, which comprises a hollow gas duct resembling the shape of a hollow tube, solid portions connecting the hollow ducts and central through holes. Blow-molding is the preferred way to produce the component and, e.g., the reflector body, since blow-molding is a low-cost process with short manufacturing times. This is achieved as blow molding is a process whereby the component is produced as an integrated one-piece component and assembly of several components is therefore avoided, while the quality of the component and the structure thereof can be maintained.

Also, preferably, the material is such that it is easy to cut after blow-molding, e.g., by means of a punching tool. Furthermore, in use it is desirable that the component is easy to connect to other components of the breathing system. Therefore, the plastic material preferably has mechanical properties tolerating sudden impacts and mechanical treatment. The material should be sufficiently rigid but not fragile, and cutting of the component with a punch tool is suitably possible. Also, it is desirable that machining and turning of the component can be performed to achieve sealing surfaces.

It has been noted that a plastic material having a flexural modulus of from 900 to 2000 MPa, or from 1000-1500 MPa, measured at 23° C. and according to ISO 178, more specifically according to the version ISO 178:2010/AMd 1:2013 valid at the priority date of the present application, has rigidity that is advantageous while still easy to handle.

Further, the breathing system component may have an Izod impact of the plastic material of at least 3 kJ/m², or from 3.5 to 10 kJ/m², measured according to ISO 180/1A, when notched at −20° C. The version of the ISO 180/1A used for the determination of the Izod impact is the version valid at the priority date of the application.

Different plastic materials may be used, but according to an aspect the plastic material may be a polyolefin-based polymer, such as polyethylene and/or polypropylene materials and/or copolymer materials thereof. The plastic material is suitably of high purity, such as more than 95% w/w, or 95% w/w or 99% or above and up to 100% w/w. Thus, the amount of additives is kept low. The density of the plastic material may vary. For example, the density may vary from 0.8 to 1.0 g/cm³ at 25° C., such as from 0.85 to 0.97 g/cm³ at 25° C., or from 0.85 to 0.94 g/cm³ at 25° C. The material may be transparent, semi-transparent or opaque. The material may be produced with a non-phthalate based catalyst.

The polymeric material may comprise additives, such as calcium stearate, antioxidants, such as phenol-based antioxidants, and, e.g., stabilizers. Other additives may be used to control the properties of the material and, for example, the level of impurities.

According to the present disclosure, the breathing system component may be used in different types of breathing systems adapted to provide a breathing gas to a patient. The breathing system may be a re-breathing system in which exhaled gases are returned to a patient. The breathing system comprises one or more breathing system components. The component of the present disclosure is a breathing system component comprising at least one hollow gas duct. The gas duct is adapted for conveying the breathing gas, which may include an inspiratory gas, expiratory gas and/or driving gas in the breathing system.

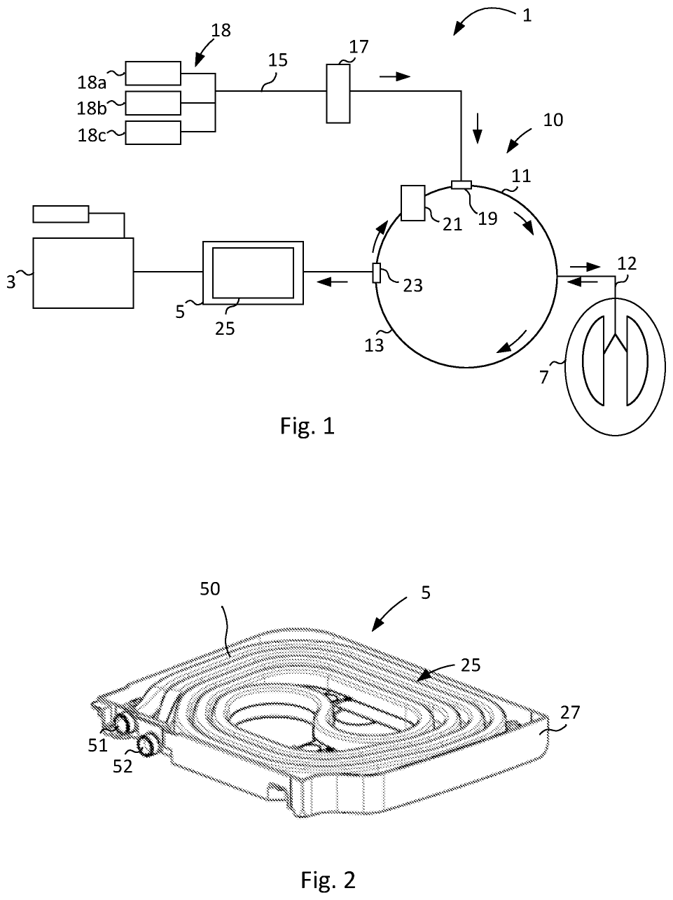

An example of a breathing system is schematically shown in FIG. 1. An example of a breathing system component, which is a volume reflector body, is shown in FIG. 2-4.

The breathing system is generally depicted by a reference number 1 in FIG. 1. The breathing system 1 comprises a ventilator 3, which generates a gas flow in the breathing system 1. The ventilator 3 is fluidly connected to a patient circuit 10 comprising an inspiratory line 11 and an expiratory line 13, which are connected to a patient depicted by reference sign 7. The patient circuit 10 is adapted to convey inhalation and expiration gas to and from the patient 7, and comprises several components comprising hollow gas ducts, such as rigid and flexible tubes, check valves and other components necessary to control the gas flow in the breathing system.

A Y-piece 12 and a patient interface (not shown) are usually used to fluidly connect the inspiratory line 11 and the expiratory line 13 of the patient circuit 10 to the patient 7. The interface may include different components such as, but not limited to, larynx tube, tracheal tube, mask etc. The inspiratory line 11 is connected to a fresh gas supply line 15 through which a fresh patient gas mixture is supplied to the patient. The patient gas mixture may comprise several gases supplied via different gas modules 18a, 18b and 18c adapted to deliver desired gases to the breathing system. Each of the gas modules may comprise a gas regulating valve, pressure and flow measuring devices and electronic devices to control the flow. For example, the first gas module 18a may be adapted to supply nitrous oxide (N₂O), the second gas module 18b may be adapted to supply air, and the third gas module 18c may be adapted to supply oxygen (O₂) to the patient circuit 10. Additionally, an anesthesia agent, e.g., halothane, enflurane, isoflurane, sevoflurane, and/or desflurane, may be supplied to the patient gas mixture from a vaporizer 17.

The gases are collected in a manifold 18 in which the gases are mixed and the gas is conveyed via the fresh gas supply line 15 to the inspiratory line 11 in the patient circuit 10. Optionally, a vaporizer 17, which may be an electronic injection type vaporizer, is connected to the fresh gas supply line 15 downstream of the manifold 18 and upstream of a fresh gas inlet 19 connecting the fresh gas supply line 15 to the inspiratory line 11.

When the expiratory gases are to be rebreathed, they are returned to the inspiratory line via a carbon dioxide absorber 21 before returning the gas to the patient. The flow direction of the expiratory and inspiratory gases can be regulated, for example, by means of check valves, compressors, fan ejectors or any other means that can regulate the flow direction.

The breathing system 1 comprises also a separation arrangement by which the driving gas of the ventilator 3 is separated from the patient breathing gases flowing in the patient circuit 10. The ventilator 3 is connected to the patient circuit 10 by means of a ventilator inlet 23, and the separator is arranged in between the ventilator and the ventilator inlet 23. The separation arrangement could be a bellows membrane in a bag-in-bottle system. However, in the shown example system of FIG. 1, a volume reflector unit 5 is used as a separation arrangement, and the function thereof is described below. The volume reflector unit comprises a volume reflector body 25 and a carrier or carrier means 27, for example in the form of a tray, adapted to carry or house the volume reflector body 25 (see FIGS. 2 to 4).

The volume reflector unit 5 fluidly separates the patient circuit 10 from the ventilator 3. The separation is an open separation, i.e., without any separating means such as membranes, in which the two adjacent gas fronts are comprised, and wherein the driving gas and the expiratory/patient gases are separated. In the embodiment further shown in FIG. 2-4, the reflector body 25 comprises a gas duct 50 adapted to separate the gases. The separation can be obtained by arranging the length of the gas duct 50 such that it is sufficiently long to be able to separate the driving gas and the expiratory gases and may comprise curved sections as further described below. The gas duct 50 provides a continuous gas duct between the ventilator driving gas supply and the patient circuit 10. The separation of the ventilator gas and the breathing gas is made with a gas gradient reciprocating in the gas flow channel 50 in a known manner whereby diffusion of driving gas and patient gas may occur. During inhalation the driving gas supply from the ventilator 3 pushes the inspiration gas towards the patient 7 and to the inspiration line 11 of the patient circuit 10. During expiration, the expiration gas is brought towards the volume reflector, whereby pressure may be released from the reflector unit 5. Thus, the volume reflector unit 5 is adapted to perform the same function as a bag-in-bottle system, thus providing a temporary gas reservoir that receives exhaled breathing gases during the expiration phase and that pushes back the breathing gases during the next inspiration phase.

In the bag-in-bottle system the driving gas is separated from the patient gas with a membrane, whereas the volume reflector unit has an open separation in the form of a narrow gas front in the gas duct of the volume reflector unit. A minor mixing of driving gas and patient gas at the gas front may occur, but that is not significant. However, by means of a volume reflector several advantages are obtained. For example, the risk for pressure drop in the patient circuit is minimized in case of gas leakage, which may occur anywhere in the system, e.g., between the patient and the patient interface. A gas leakage in a bag-in-bottle system may cause the bellows to drop to the bottom of the bottle if the combined tidal volume and leakage volume is bigger than the volume contained in the bellow, whereby pressure drop occurs in the patient circuit. Since the volume reflector has an open structure, such pressure drop will not occur as the driving gas will compensate for any lost breathing gas and maintain pressure in the patient circuit. In this case, a minor dilution of the supplied breathing gases will occur that is easily compensated for, and there is a reduced risk for alveolar collapse. In addition, by not using a membrane between the driving gas and breathing gas, the precision of the delivered gas pressure/flow is significant.

Generally, the reflector body comprises a hollow gas duct for conveying an inspiratory gas to a patient. The patient circuit is allowed to use the reflector body volume constituted by the gas duct of the reflector body for a driving gas pillar, which virtually moves back and forth in the reflector volume. The reflector volume is cyclically filled with previously exhaled gas, e.g., comprising an anesthetic gas, which is returned to a patient circuit for re-use, i.e., re-breathing, during a subsequent inspiration. The driving gas for the volume reflector, usually oxygen or air, is used as the driving gas pillar pushing the patient gas pillar back into the patient circuit towards the patient during inspiration. Upon the subsequent expiration, the reflector volume is re-filled with expiratory gas and the driving gas pillar is pushed out of the reflector volume towards a gas evacuation system, EVAC. EVAC is usually present in operating theatres and connected to the exhaust of anesthesia machines for taking care of waste gases to avoid anesthetic gases escaping into the surrounding environment. In this manner, an adjacent patient gas pillar is alternatingly virtually moving out of the patient circuit into the reflector volume during exhalation, and back into the patient circuit via the carbon dioxide absorber from the reflector volume during inspiration.

Returning to FIG. 2-4, the volume reflector body 25 and the gas duct 50 thereof have a fixed and pre-determined volume. The reflector body has an integrated one-piece molded structure and is made of a non-toxic plastic material. Thereby, the reflector body can be fitted into, e.g., an existing breathing apparatus. Also the gas duct can be cyclically filled with the driving gas and the expiratory gas without mixing or with minimized mixing of the gases, suitably according to pre-determined parameters. The length of the gas duct may be arranged to be suitable for the patient in question, e.g., for an adult or infant patient. The size of the carrier or carrier means may be the same for all reflector body volumes, whereby it is possible to fit the reflector body to the existing apparatus regardless of the size or volume of the reflector body.

The gas duct 50 of the reflector body 25 comprises at its respective free end a first port 51 and a second port 52. The respective port 51, 52 may comprise means to connect the component to the breathing system 1, i.e., a connector, in order to connect respectively port 51 to a line conveying driving gas from the ventilator and port 52 to a line conveying expiratory gas from the patient. The carrier means 27 may comprise openings or recesses for the ports 51 and 52. In the shown example, the patient circuit 10 is fluidly connected to the second port 52, such that the expiratory gas can be received during expiration via the second port 52 while gas in the volume reflector 5 is pushed through the first port 51 as a waste gas flow to an exhaust of the breathing system. The driving gas from the ventilator 3 is connected to the first port 51 and during inspiration the driving gas pillar pushes the patient gas pillar back into the patient circuit 10. Expired gas from a preceding expiration is thus re-breathed by the patient in subsequent inspiration.

The volume reflector body 25 may be designed in various ways in order to provide a well-defined gas front, and the gas duct 50 or tube of the volume reflector is preferably chosen to be narrow. However, this dimension may be weighed against increased flow resistance. Also, compressible volume is chosen to be as low as possible. Preferably, the total volume of the volume reflector body is as close to the average tidal volume of the patient as possible. The gas duct may have a substantially circular cross-section or it may be rectangular with rounded corners or it may be oval or nearly oval. The cross section area of the duct, i.e., in the plane perpendicular to the flow direction, is suitably from 300 to 450 mm², suitably from 350 to 400 mm², and preferably about 370 mm². The length of the gas duct may be, for example, from 0.5 to 4 m and can be adapted to the patient in question, e.g., an infant or an adult. Likewise, the total volume of the gas duct may be adapted to the patient in question, e.g., an infant or an adult and can be, for example, from 0.1 to 2 litres or from 0.2 to 1.5 litres, preferably from about 1.0 to 1.5 litres.

The reflector body 25 according to the present disclosure has an integrated one-piece molded structure. The hollow gas duct 50 may be a single continuous gas duct comprising straight and curved portions. The hollow gas duct 50 may be arranged such that at least two gas duct sections are located adjacent to each other. The sections may be connected to each other by means of at least one solid portion between the sections.

The reflector body 25 has an outer extension defined by the outer edges of the reflector body. The outer edges may define in an X-Y plane a shape resembling approximately a quadrilateral shape with rounded corners. The shape is not limited thereto, and it could be substantially circular, oval or rectangular, preferably comprising at least one rounded corner. By a shape resembling approximately a quadrilateral shape with rounded corners design it can be easily fitted into existing equipment. The reflector body 25 may be fitted to a carrier means 27 also having an inner shape resembling the shape of the reflector body 25. It can thus be a quadrilateral shape with at least one rounded edge. The carrier or carrier means, which may be in the form of a tray, thus comprises a cavity for carrying or housing the reflector body and the carrier may comprise side walls at least partially surrounding the reflector body. Thus, the carrier is adapted for keeping the reflector body in its position.

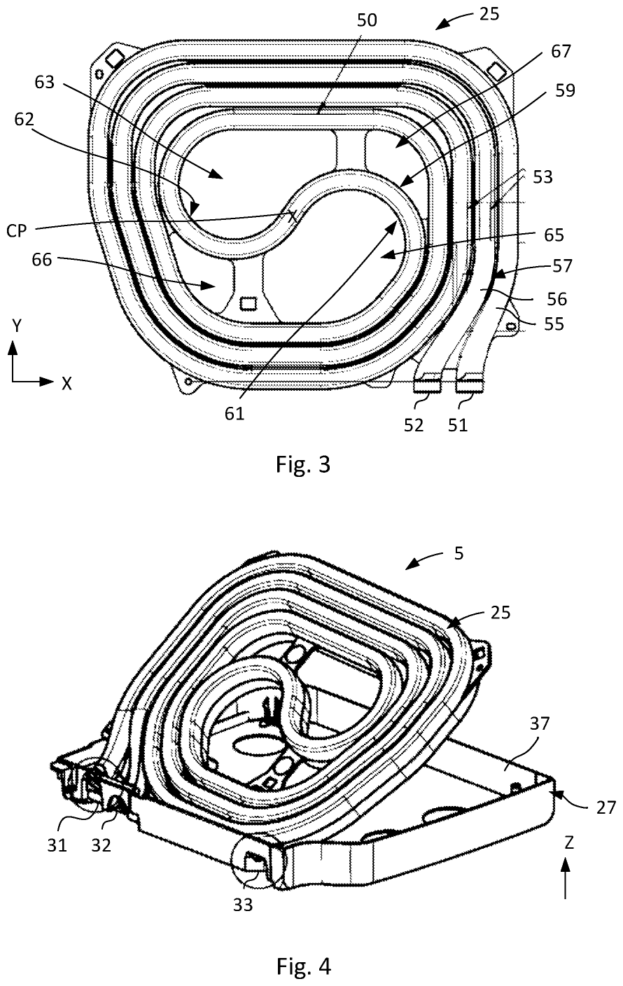

The X-Y plane is defined as a two-dimensional plane when the reflector body/carrier means is viewed directly from above or below, and the plane is shown in FIG. 3, in which the reflector body 25 is shown from below. A Z-plane is defined as a plane transversal to the X-Y plane and showing the height dimension of the reflector body/carrier means when the reflector body/carrier means is viewed from the side, and the Z-plane is shown by an arrow in FIG. 4.

Reference is made to FIG. 3, which shows an example embodiment of the volume reflector body 25 from below, wherein the hollow gas duct 50 of the reflector body may be arranged so that it has a gas duct 50 shape resembling a shape of a folded tube. The gas duct 50 has a substantially rectangular cross section with rounded corners as indicated by reference numbers 53. The gas duct 50 may be folded such that the free ends comprising a first port 51 and a second port 52 of the duct are placed adjacent to each other and the at least two adjacent gas duct portions 55, 56, in proximity of respective free end, run side-by-side. The free ends of the tube are placed in a parallel manner in the X-Y plane. The shape of the reflector body 25 resembles a shape in which the duct 50 is spirally wound inwards towards a centre point CP of the reflector body. The folded end 59 of the gas duct is arranged such that two central loops 61 and 62 are formed in the centre portion of the quadrilateral shape. The duct sections running in parallel, i.e., the sections 55 and 56, may be connected to each other by means of a solid portion 57 arranged in between the sections. The folded end portion of the gas duct may be formed so as to define the loop portions 61 and 62 and so that openings 63 and 65, which are through holes when viewed in X-Y plane, are formed. The reflector body may optionally comprise at least one additional through hole 66, when viewed in X-Y plane. The embodiment shown in FIG. 3 comprises a further through hole 67. In this way, a lighter construction for the reflector body can be obtained.

In an alternative embodiment, the free ends comprising the first port and the second port of the duct can be placed in a manner where they are not adjacent to each other. For example, in an alternative embodiment, which is not shown, the gas duct could be arranged in wave-form and so that the free ends of the duct are not arranged adjacent to each other, but instead at opposite ends of the wave-formed gas duct.

The breathing system may comprise further components, e.g., controlled inspiratory or expiratory valves, check valves, flow sensors, pressure sensors, tubes and connectors. The tubes may be rigid or flexible and contain a hollow gas duct adapted to convey gas in a breathing system. For example, the inspiratory and expiratory lines may be rigid tubes, while the Y-piece may be flexible, or vice-versa, or in some embodiments, they could all be flexible or rigid. The breathing system component according to the present disclosure could also be a gas supply line conveying gas between the ventilator and the patient circuit or between the gas modules and the patient circuit. For example, the breathing system component according to the present disclosure could be any one of the breathing gas conveying components of FIG. 1, for example, the fresh gas line 15. However, the implementation is more complex than the schematic sketch of FIG. 1, with further components included, for example, the manifold structure and all the interface parts with connections to the gas modules 18a, 18b, 18c and the vaporizer 17. Furthermore, the breathing circle 10 could partly or fully be manufactured according to the disclosure. In addition, the expiratory cassette (not shown) comprising the expiratory valve flow tranducers could be a breathing system component according to the present disclosure. Both the expiratory cassette and the breathing circuit 10 are configured to be in contact with contaminated exhaled patient gas and, therefore, these components preferably also withstand high cleaning temperatures including autoclaving.

As illustrated in FIG. 4, the reflector unit 5 comprises the reflector body and a carrier 27, which has a shape and size adapted to keep the reflector body in place during the operation of the breathing apparatus. The carrier may have a standard size while the size and shape of the reflector body 25 may vary. The carrier may comprise adapter means arranged to, e.g., lock or otherwise keep the reflector body in place. The carrier means also comprises openings 31 and 32 for respective ends 51 and 52 of the gas duct 50. The carrier means may comprise additional opening 33 arranged to receive different connectors, sensors or devices from the breathing apparatus. The opening may be also formed as a cavity or recess, and is adapted to receive different connectors, sensors or devices from the breathing apparatus. The carrier means 27 has edges 37 adapted to at least partially surround the volume reflector body 25. The carrier means may be made of a dimensionally stable material, such as plastic or metal, e.g., aluminium, preferably plastic. According to the embodiment shown in the drawings, the carrier means and the reflector body may be separate elements. However, the carrier means and the reflector body could be integrated to a one-piece construction. Furthermore, the carrier means could be omitted completely, whereby the reflector unit consists of the reflector body alone, which can then be further connected to other components of the breathing system.

According to an embodiment of the present disclosure, the breathing system component is the above-described reflector body. According to another embodiment, the component is any other component in the breathing system adapted to convey a breathing gas. The component comprises a gas duct which is in direct contact with the patient or in indirect contact with the patient. For example, the driving gas may not be in direct contact with the patient, e.g., under normal leakage free circumstances, but is instead used to push an inspiratory gas to the patient, and the driving gas is thus in indirect contact with the patient.

The breathing system component, such as the reflector body defined above, may be produced by means of blow molding. By blow molding is meant a process for forming plastic objects in which a plastic material, i.e., a thermoplastic polymeric raw material, is melted, put in a mold, and then shaped by having compressed air blown into it. The compressed air may be blown into the material, for example, by means of a blow needle inserted into the material. In another variant of the process, the plastic material can be melt down and then pre-formed to an initial form, which is often referred to as a pre-form or a parison. The parison may comprise an opening through which gas, e.g., air, can pass. The parison is then clamped into a mold and compressed air is blown into the structure and thus the polymeric material is pressurized. In this way the thermoplastic polymeric material is pressed towards the contours of the mold resembling the final shape of the breathing system component, e.g., the reflector body. After a pre-determined or desired blowing of the compressed air, the material is allowed to cool down and harden or cure. Subsequently, the mold is opened and the reflector body is removed or automatically ejected by means of an ejection device from the mold. The reflector body may be additionally cut to remove excess material.

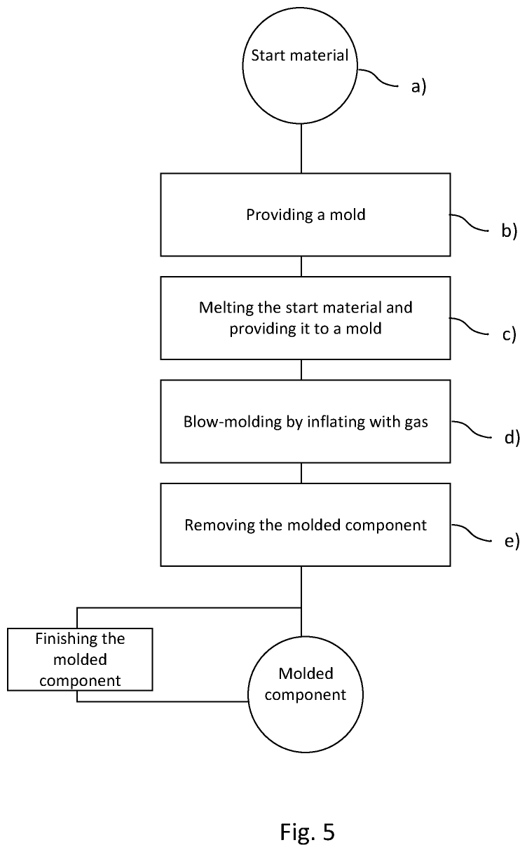

Reference is made to FIG. 5 showing a flow chart for an example process for the manufacture of a breathing system component comprising a hollow gas duct adapted for conveying an inspiratory gas to a patient or an expiratory gas from a patient.

In step a), a non-toxic plastic material having a melt flow rate MFR of 2.5 g/10 min or less, measured according to ISO 1133-1:2011 with 2.16 kg/230° C. for the component is provided. The material is suitable for blow-molding and is described more in detail above.

In step b), a mold is provided. The mold comprises a cavity having a shape corresponding to the shape of outer contours of the breathing system component.

In the next step c), the non-toxic plastic material is melted and a parison of the molten plastic material is formed. A parison is a hollow tube consisting of the moldable material which is blow-molded in the subsequent step d). The parison may be provided, for example, by means of extrusion or injection molding, e.g., by melting the plastic material in an extruder and by pressing the material through a nozzle to form the parison. The material in the parison is viscous and rubbery, the grade of which is dependent on the MFR-value of the material.

In step d), the component is blow molded by means of inflating the parison with pressurized gas, such as air, so that it is pressed towards the walls of the cavity in the mold.

In the final step e), the mold is opened and the blow-molded component is removed. The component is finally cured either in the mold or after removal from the mold. The blow-molding process may be performed in existing blow-molding systems. The specific MFR of the plastic material makes it possible to blow mold also structures comprising parallel duct sections, which may be curved and/or straight.

Additionally, the process may comprise cutting the blow molded component to remove excess material surrounding the edges of the component. Also other steps may be included to obtain a desired final shape or surface features for the component. For example, in a process where the manufactured component comprises two free gas duct ends, the process may comprise a step of machining the free ends to provide sealing surface for additional components.

### EXAMPLES

Materials Used

Sample 1 is a commercially available, proprietary thermoplastic polyester elastomer (KOPEL® KP3956BM, Kolon Plastics Inc.). It contains a polyether soft segment and a hard polyester segment. The material possesses good mechanical strength, flexibility at low temperature and high elasticity. It also exhibits good heat-, creep-, chemical-, high temperature-, fatigue- and weather resistance. It contains Additive KY-405 as heat oxidative stabilizer. The material is not graded as food grade.

Sample 2 plastic corresponds to the material of Sample 1 plastic, but was modified with different heat stabilizer to achieve lower leachable/extractable levels, and is thus of food grade. More specifically, Sample 2 is a commercially available, proprietary thermoplastic polyester elastomer (KOPEL® KP3956BM FC, Kolon Plastics Inc.). It contains a polyether soft segment and a hard polyester segment. Sample 2 plastic has the same general properties as the Sample 1 plastic characterized above. Sample 2 contains additives Irganox 1010 and Irganox 1098 as heat oxidative stabilizers.

Sample 3 is a commercially available, proprietary polymethylpentene (TPX DX845, Mitsui Chemicals Inc.). It is a thermoplastic in the polyolefin family. It has high transparency. The material possesses excellent heat- and chemical resistance. It shows very low water absorbance and is compatible with steam sterilization. The material is of food grade.

Sample 4 is a commercially available, proprietary polypropylene-ethylene copolymer. It has good cold impact resistance and stiffness, is suitable for blow molding applications and can be steam steam-sterilized or autoclaved. It is produced with a non-phthalate based catalyst. The material is of food grade.

A summary of the properties for the materials is listed in Table 1 below.

TABLE 1
Samples Materials for volume reflector

| | | MFR | | | | | |
|---|---|---|---|---|---|---|---|
| | | g/10 min | | | | | |
| | | 230° C./2.16 | Melt | | | | |
| | | kg, ISO | point | | | Post | |
| Sample | | 1133- | DSC | | | processing | Food |
| # | Material type | 1:2011 | C. ° | Color | Test article | treatment | grade |
| 1. | Thermoplastic | 1.5 | 220 | White | Blow molded | No | No |
| | Copolyester | | | | sample shape | | |
| | Elastomer | | | | | | |
| 2. | Thermoplastic | 1.95** | 220 | White | Blow molded | No | Yes |
| | Copolyester | | | | | | |
| | Elastomer with | | | | | | |
| | modified | | | | | | |
| | stabilizer | | | | | | |
| 3. | 4- | 9* | 232 | Trans- | Blow molded | No | Yes |
| | methylpentene-1- | | | parent | sample shape | | |
| | based olefin | | | | | | |
| | copolymer | | | | | | |
| 4. | Polypropylene- | 0.8 | 164 | Semi- | Blow molded | No | Yes |
| | ethylene | | | trans- | | | |
| | copolymer | | | parent | | | |

*Measured at 260° C./5 kg
**Measured at 230° C./5.00 kg

### Example 1—Mechanical Properties

In the present example, materials having different melt flow rates were used to blow mold a reflector body shown in FIG. 2-4. Blow molding was performed in a conventional blow molding apparatus and the process was performed in accordance with the process defined in the description.

The mechanical properties of the materials were evaluated based on the suitability for the manufacturing process and the properties of the reflector unit produced. The following conclusions were made:

It was possible to blow mold Samples 1 and 2 but both were more difficult to machine with sealing surfaces.

Sample 3 was not consistent in melting together around the edges of the component. Also it was judged to be too brittle for the cutting punch tool and sensitive for cracking in the end product.

Of the evaluated materials, the material of Sample 4 was the most suitable for all steps of the manufacturing including blow molding, cutting of the component with a punch tool and machining and turning.

### Example 2—Chemical Resistance

Blow molded samples 2 and 4 manufactured with the materials specified in Table 1 above were tested to assess the possibility to wash out the residue of anesthetic agent after exposing the samples to high levels of the anesthetic agent. The samples were assembled into an anesthesia machine (FLOW-i® by Maquet) and exposed to high levels of Isoflurane. After exposure and following recommended cleaning procedure, the time required to achieve less than 5 ppm of Isoflurane in the fresh gas flow relevant for infant patient flow rates through the object was measured. For both Sample 2 and Sample 4 the time required was less than 120 minutes. Thus, both materials have an acceptable level of chemical resistance.

### Example 3 Suitability for Steam Sterilization

General properties

Samples 1 and 2

The material is graded to be compatible with steam sterilization.

Sample 3

Generally, polymethylpentene materials withstand repeated autoclaving up to 150° C.

Sample 4

The polypropylene material is graded to be suitable for sterilizing at 121° C.

Test Method

Initial tests of blow molded samples of Sample 1 and Sample 3 after single autoclaving cycle at 131° C. were made; discoloration was observed for Sample 3 and a slight discoloration for Sample 1.

Sample 4 also showed a slight discoloration after 23 cycles exposure of medical washer-disinfector, medical drying cabinet and sterilizer.

None of the tested samples showed any change of dimension or mechanical rigidity that effects the objects intended use.

The discoloration is considered as acceptable by product definition group including clinicians.

### Example 4 Volatile Extractable

Test Method

Granulates of the materials of the blow molded samples 1-4 were extracted in 20% ethanol or acetonitrile at 50° C. for 72 hrs. An extraction ratio of 3 cm²/ml or 0.2 g/ml was used. The extracts were analyzed using direct inject gas chromatography/mass spectrometry (GC/MS) for analysis of volatile substances.

The maximum duration of use of a volume reflector is normally limited to be less than 24 h. However, a 72 hour extraction time was therefore chosen to also cover cases of longer use.

GC/MS is used for identifying and assessing semi-volatile extractables such as residual monomers, antioxidants, plasticizers, antistatic agents, clarifying agents, preservatives and slip agents.

The total levels of the volatile extractables are listed in Table 4. Some of the results were recalculated from total extractables per weight to total extractables per surface area for comparative purposes. Highest levels of volatile extractables were observed for Sample 1, which is not a food grade plastics material, where almost the entire amount of extractables originated from the anti-oxidant used in the material. All other materials, which are food grade plastic materials, had low levels of extractable volatiles.

TABLE 4
Total level of volatile extractables

| | Total level of extractables |
|---|---|
| Sample material | [μg/cm²] |
| Sample 1 | 290¹⁾ |
| Sample 2 | 2.2²⁾ |
| Sample 3 | ND¹⁾ |
| Sample 4 | ND²⁾ |

¹⁾Extraction performed in acetonitrile
²⁾Extraction performed in 20% EtOH

A summary of the rating for the different material candidates are summarized in Table 5. Sample materials 2 and 4 are the most suitable materials for the volume reflector.

TABLE 5
Summary of material candidate properties

| | Mechanical | | | | |
|---|---|---|---|---|---|
| | and | | Suitability | | |
| | processing | Chemical | for steam | Volatile | Suitable |
| Sample# | properties | resistance | sterilization | extractables | candidate |
| 1. | Satisfactory | Satisfactory | Satisfactory | Not satisfactory | No |
| 2. | Satisfactory | Satisfactory | Satisfactory | Satisfactory | Yes |
| 3. | Not Satisfactory | Satisfactory | Satisfactory | Satisfactory | No |
| 4. | Satisfactory | Satisfactory | Satisfactory | Satisfactory | Yes |

While this disclosure provides multiple exemplary embodiments, it will be understood by those skilled in the art that various changes may be made and equivalents may be substituted for elements thereof without departing from the scope of this disclosure. In addition, many modifications may be made to adapt a particular situation or material to the teachings of this disclosure without departing from the essential scope thereof. Therefore, it is intended that the invention, as defined in the appended claims, not be limited to any particular embodiment disclosed herein, but that the invention will include all embodiments falling within the scope of the claims. Also, in the drawings and the description, there have been disclosed exemplary embodiments and, although specific terms may have been employed, they are unless otherwise stated used in a generic and descriptive sense only and not for purposes of limitation. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item unless otherwise explicitly indicated.

1. A breathing system component comprising:
a hollow gas duct that conveys breathing gas in a breathing system, wherein the breathing system component has an integrated one-piece molded structure and is made of a non-toxic plastic material having a melt flow rate (MFR) of 2.5 g/10 min or less, measured according to ISO 1133 with a load of 2.16 at a temperature of 230° C.

2. A breathing system component according to claim 1, wherein the melt flow rate (MFR) is from 0.6 g/10 min to 1.0 g/10 min, measured according to ISO 1133 with a load of 2.16 kg at a temperature of 230° C.

3. A breathing system component according to claim 1, wherein the breathing system component is molded by blow-molding.

4. A breathing system component according to claim 1, wherein the plastic material has a melting point adapted to be above a sterilization temperature of the breathing system component and the melting point is at least 134° C. measured by differential scanning calorimetry according to ASTM D3418-15.

5. A breathing system component according to claim 1, wherein the flexural modulus of the plastic material is from 900 to 2000 MPa measured at 23° C. and according to ISO 178.

6. A breathing system component according to claim 1, wherein the density of the plastic material is from 0.8 g/mL to 1.0 g/mL at 25° C.

7. A breathing system component according to claim 1, wherein the plastic material is polyolefin-based.

8. A breathing system component according to claim 7, wherein the polyolefin-based material is polyethylene, polypropylene or a copolymer thereof.

9. A breathing system component according to claim 1, wherein the hollow gas duct is a single continuous gas duct comprising straight and curved portions.

10. A breathing system component according to claim 9, wherein the hollow gas duct is arranged such that at least two gas duct sections are located adjacent to each other and connected to each other by at least one solid portion.

11. A breathing system component according to claim 9, wherein the gas duct comprises free ends, wherein at one free end is formed a first port and at a second free end is formed a second port, and each of the first port and the second port comprises a connector to connect the breathing system component to the breathing system.

12. A breathing system component according to claim 11, wherein the hollow gas duct has a shape of a folded tube in which the free ends of the gas duct are placed adjacent to each other and wherein at least two adjacent gas duct portions run side-by-side, and wherein the tube is spirally wound inwards towards a centre portion of the breathing system component.

13. A breathing system component according to claim 12, wherein a folded end of the tube is arranged so that two central loops are formed in the centre portion of the breathing system component.

14. A breathing system component according to claim 1, wherein the breathing system component has a substantially quadrilateral shape.

15. A breathing system component according to claim 1, wherein the breathing system component is a volume reflector body.

16. A breathing system component according to claim 15, wherein the length of the gas duct in the volume reflector body is from 0.5 m to 4 m.

17. A breathing system component according to claim 16, wherein the total gas duct volume is from 0.1 litres to 2 litres.

18. A process for the manufacture of a breathing system component of claim 1, the process comprising the steps of:
(a) providing a non-toxic plastic material having a melt flow rate (MFR) of 2.5 g/10 min or less, measured according to ISO 1133 with load of 2.16 kg at a temperature at 230° C.;
(b) providing a mold comprising a cavity having a shape corresponding to the shape of an outer contour of the breathing system component;
(c) melting the non-toxic plastic material and providing the melted plastic material to the mold;
(d) blow molding the breathing system component by inflating the melted plastic material with pressurized gas so that the melted plastic material is pressed towards the walls of the cavity in the mold; and
(e) opening the mold and removing the breathing system component from the mold.

19. A process according to claim 18, wherein step (c) includes a step of forming a parison of the molten plastic material, and wherein step (d) includes a step of inflating the parison with pressurized gas.

20. A process according to claim 19, wherein the parison is formed by extrusion or is pre formed by injection molding.

21. A process according to claim 18, further comprising the step of:
cutting the blow molded component to remove excess material surrounding the edges of the breathing system component.

22. A process according to claim 18, wherein the breathing system component comprises two free gas duct ends, and the process further comprises the step of:
machining the two free gas duct ends to provide sealing surfaces for additional components.

23. (canceled)

24. A volume reflector unit comprising the breathing system component of claim 15 and a carrier having a shape adapted to the shape of the volume reflector body.

25. The volume reflector unit according to claim 24, wherein the carrier has edges adapted to at least partially surround the volume reflector body.

26. A breathing system comprising a ventilator providing a driving gas flow, a patient circuit comprising an inhalation line and an exhalation line connectable to a patient, a fresh gas supply inlet connectable to the inhalation line, and an arrangement that separates the driving gas provided by the ventilator from the patient breathing gases in the patient circuit, wherein the breathing system comprises the breathing system component according to claim 1.

27. A breathing system component according to claim 4, wherein the melting point of the plastic material is from 140° C. to 220° C. measured by differential scanning calorimetry according to ASTM D3418-15.

28. A breathing system component according to claim 1, wherein the flexural modulus of the plastic material is from 1000-1500 MPa, measured at 23° C. and according to ISO 178.

29. A breathing system component according to claim 15, wherein the breathing system comprises a ventilator.
